(19) United States
(12) Patent Application Publication
(10) Pub. No.: US 2003/0228293 A1
(43) Pub. Date: Dec. 11, 2003

(54) Correction of genetic defects

(75) Inventors: William Rideout, Cambridge, MA (US); Konrad Hochedlinger, Cambridge, MA (US); Michael Kyba, Cambridge, MA (US); Rita Perlingeiro, Cambridge, MA (US); George Q. Daley, Weston, MA (US); Rudolf Jaenisch, Brookline, MA (US)

Correspondence Address: ROPES & GRAY LLP, ONE INTERNATIONAL PLACE, BOSTON, MA, 02110-2624, US

(73) Assignees: Whitehead Institute for Biomedical Research, Cambridge, MA; The General Hospital Corporation, Boston, MA

(21) Appl. No.: 10/384,361

(22) Filed: Mar. 7, 2003

Related U.S. Application Data
(60) Provisional application No. 60/362,961, filed on Mar. 8, 2002.

(51) Int. Cl. A61K 48/00; C12N 5/08; C12N 15/85

(52) U.S. Cl. 424/93.21; 435/455; 435/366

(57) ABSTRACT

A method of correcting of treating a genetic disorder by combining therapeutic cloning and gene therapy.

### RELATED APPLICATION

This application claims the benefit of the filing date of U.S. provisional application No. 60/362,961, entitled “Correction of Genetic Defects,” filed Mar. 8, 2002. The entire teachings of the referenced application are expressly incorporated herein by reference.

### GOVERNMENT SUPPORT

Work described herein was funded, in whole or in part, by National Institutes of Health Grants CA 86991 and DK 59279, as well as National Cancer Institute Grant 5-R37-CA84198.

### BACKGROUND OF THE INVENTION

Treatment of genetic disorders, which affect the lives of many individuals, presents many challenges and typically includes approaches such as bone marrow transplantation, long term administration of drugs or both. Additional approaches to treating genetic diseases are needed.

### SUMMARY OF THE INVENTION

Nuclear transfer technology enables the reprogramming of a somatic cell into an embryonic stem cell line that can then be used to generate replacement tissues and cell types that are genetically matched to the recipient. As described herein, therapeutic cloning or nuclear transplantation therapy is useful to treat (prevent, correct or reverse) a wide variety of conditions and diseases, both genetic and acquired. The present invention relates to methods of correcting a genetic defect in an individual in need thereof; methods of treating (preventing, correcting, reversing or reducing the extent of) a condition, such as a genetic or acquired condition, in an individual in need thereof; and cells, referred to as repaired ntES cells useful in the claimed methods. The invention further relates to methods of producing repaired ntES cells and pharmaceutical compositions comprising repaired ntES cells. The methods and repaired ntES cells of the present invention are useful for treating a wide variety of conditions, such as hematopoietic conditions (e.g., sickle cell anemia, leukemias, immune deficiencies), cardiac disorders (e.g., myocardial infarcts, and myopathies) and disorders such as liver disease, diabetes, thyroid abnormalities, neurodegenerative/neurological disorders (e.g., Parkinson's, Alzheimer's, stroke injuries, spinal chord injuries), circulatory disorders, respiratory disorders and enzyme abnormalities.

In one embodiment, this invention is a method of treating a genetic defect in an individual in need thereof, comprising administering to the individual a therapeutically effect amount of repaired ntES cells, repaired differentiated progenitor or precursor cells derived from repaired ES cells or both. As used herein the term “repaired ntES cells' also encompasses repaired differentiated progenitor or precursor cells derived therefrom. The genetic defect to be treated in the individual (e.g., a genetic defect that causes immune deficiency in the individual) has been corrected in the repaired ntES cells administered to the individual, using known methods, such as a recombinant nucleic acid (DNA, RNA) method, such as homologous recombination, small interfering RNA (siRNA) or microRNA (miRNA) methods. In this embodiment and all other embodiments, the genetic defect can be inherited/congenital or acquired, such as a result of environmental damage. A therapeutically effective amount of repaired ntES cells is sufficient to correct a genetic defect or treat (prevent, correct, reverse or reduce) a condition caused or contributed to by a genetic defect. A therapeutically effective amount can be administered in one or more doses. The number of doses and the time over which they are administered will depend in part, on the genetic defect being corrected or the condition being treated. A single dose of repaired ntES cells or multiple doses can be administered; if multiple doses are administered, they can be administered at regular intervals (e.g., one or more times daily, weekly, monthly) or on an as-needed basis. Repaired ntES cells can be administered in a pharmaceutical composition which comprises, for example, an appropriate carrier (e.g., a physiologically acceptable buffer). They can be administered in a regimen which also includes administration of at least one additional therapeutic or prophylactic agent, or procedure, such as a drug, irradiation or a surgical procedure. In specific embodiments, the genetic defect that is corrected is one which causes an immune system disorder, a neurological disorder, a cardiac disorder, a circulatory disorder or a respiratory disorder.

In another embodiment, the present invention is a method of treating a genetic disorder in an individual in need thereof, comprising administering to the individual a therapeutically effective amount of repaired ntES cells, repaired differentiated progenitors or precurser cells or both (collectively, repaired ntES cells). Repaired ntES cells administered in this embodiment are repaired cells in which a defect(s) in a gene or genes that causes or is associated with the genetic disorders has been corrected. A therapeutically effective amount of repaired ntES cells is sufficient to treat (prevent, correct, reverse or reduce) the genetic disorder. The number of doses and the time over which the repaired ntES cells are administered will depend on the genetic disorder being treated. A single dose of repaired ntES cells or multiple doses can be administered; if multiple doses are administered, they can be administered at regular intervals (e.g., one or more times daily, weekly, monthly) or on an as-needed basis. Repaired ntES cells can be administered in a regimen which also includes administration of at least one additional therapeutic or prophylactic agent, or procedure, such as a drug, irradiation or a surgical procedure. In specific embodiments, the genetic disorder that is treated is an immune system disorder, a neurological disorder, a cardiac disorder, a circulatory disorder or a respiratory disorder.

Repaired ntES cells are also the subject of the present invention. Such cells are ES cells produced by correcting a genetic defect in ES cells obtained from a donor by known methods or by culturing/expanding a population of ES cells in which a genetic defect has been corrected. The term “repaired ntES cells” includes cells in which a genetic defect has been corrected, progeny thereof and differentiated precursor and progenitor cells derived therefrom. The source or donor of ES cells to be repaired (referred to as the original ES cells) can be an individual (human or nonhuman, such as dog, cat, pig, goat, cow, mouse, rat, rabbit) or tissue or cells obtained from a tissue/cell bank or repository. The repaired ntES cells can be administered to the individual from whom the original ES cells were obtained (the donor) or to another individual. In a specific embodiment, repaired ntES cells produced from cells obtained from an individual are administered to that individual.

One embodiment of producing repaired ntES cells is as follows: Somatic cells containing a genetic defect to be corrected are obtained and cultured, as needed. Somatic cell nuclei are transferred into enucleated oocytes, and the products of the transfer, referred to as modified oocytes, are cultured under conditions appropriate for (that result in) blastocyst formation. ES cells, which contain the genetic defect, are isolated and the genetic defect is corrected (e.g., by homologous recombination), resulting in production of repaired ntES cells. Such cells can be administered as described herein. Preferably, they are differentiated in vitro into tissue or cell types useful in treating the condition or disorder from which an individual (e.g., the donor of the somatic cell nuclei) suffers. Differentiation can be effected by known methods. In one embodiment of the present method, repaired ntES cells are used to produce hematopoietic stem cells (HSC) which are useful for transplantation and restoration of immune function in immune deficient recipients. In this embodiment, repaired ntES cells are differentiated into erythroid bodies (EB's) and infected with the HoxB4 gene. The resulting infected cells are cultured under conditions appropriate for (which result in) formation of HSCs, which are useful for transplantation into an immune deficient recipient.

In a further embodiment, the present invention is a method of producing repaired ntES cells, comprising: (a) introducing nuclei from a somatic cell into enucleated oocytes, wherein the somatic cell comprises DNA comprising a genetic defect; (b) maintaining the product of (a) under conditions appropriate for blastocyst formation, thereby producing blastocysts comprising DNA from the somatic cell; (c) obtaining embryonic stem cells from blastocysts produced in (b), wherein the embryonic stem cells are referred to as ntES cells and comprise DNA comprising the genetic defect; and (d) correcting the genetic defect in the ntES cells, thereby producing repaired ntES cells. The ntES cells can be, for example, mouse cells or human cells.

In further embodiments, repaired ntES cells are maintained under conditions which result in their differentiation into a desired cell type(s) (e.g., into repaired neurons, cardiac myocytes).

Repaired ntES cells, such as human repaired ntES cells and mouse repaired ntES cells, as well as repaired ntES cells of other animals (e.g., rat, cat, dog, pig, horse, goat, bird) are the subject of this invention. Such cells are administered to a recipient by an appropriate route (e.g., intravenously, intramuscularly, intraperitoneally). They can be administered in conjunction with another approach or method of treatment of the condition or disorder (e.g., in conjunction with a drug, surgery, irradiation). Pharmaceutical compositions comprising repaired ntES cells and an appropriate carrier (e.g., a physiologically acceptable buffer) are also the subject of this invention. Described herein is a model of therapeutic cloning in which the severe combined immune deficiency resulting from mutation of the Rag2 recombinase gene is corrected by combined gene and cell therapy. Immune deficient Rag2−/− mice were used as nuclear donors for transfer into enucleated oocytes and the resulting blastocysts were cultured to isolate an isogenic embryonic stem cell line. One of the mutated alleles in the Rag2−/− ES cells was repaired by homologous recombination, thereby restoring normal Rag2 gene structure. The immune disorder was treated through the use of repaired ES cells in two ways. (i) Immune competent mice were generated from the repaired ES cells by tetraploid embryo complementation, and were used as bone marrow donors for transplantation. (ii) Hematopoietic precursors were derived by in vitro differentiation from the repaired ES cells and engrafted into mutant mice. Mature myeloid and lymphoid cells as well as immunoglobulins became detectable 3 to 4 weeks after transplantation. The work described herein establishes a paradigm for the treatment of a genetic disorder by combining therapeutic cloning with gene therapy.

Also described herein is an assessment of the extent to which primitive embryonic blood progenitors contribute to definitive lymphoid-myeloid hematopoiesis in the adult. In an effort to characterize factors that distinguish the definitive adult hematopoietic stem cell (HSC) and primitive progenitors derived from yolk sac or embryonic stem (ES) cells, Applicants examined the effect of ectopic expression of HoxB4, a homeotic selector gene implicated in self-renewal of definitive HSCs. Expression of HoxB4 in primitive progenitors combined with culture on hematopoietic stroma induces a switch to the definitive HSC phenotype. These progenitors engraft lethally irradiated adults and contribute to long-term, multilineage hematopoiesis in primary and secondary recipients. These results suggest that primitive HSC are poised to become definitive HSC, and that this transition can be promoted by HoxB4 expression.

### BRIEF DESCRIPTION OF THE DRAWINGS

FIG. 1 shows a scheme for therapeutic cloning combined with gene and cell therapy. A piece of tail from a mouse homozygous for the recombination activating gene 2 (Rag2) mutation was removed and cultured. After fibroblast-like cells grew out, they were used as donors for nuclear transfer by direct injection into enucleated MII oocytes using a Piezoelectric driven micromanipulator. Embryonic stem (ES) cells isolated from the NT-derived blastocysts were genetically repaired by homologous recombination. After repair, the ntES cells were differentiated in vitro into embryoid bodies (EBs), infected with the HoxB4iGFP retrovirus, expanded, and injected into the tail vein of irradiated, Rag2-deficient mice.

FIG. 2 presents a scheme for repairing the knockout allele of Rag2. The top line illustrates the mutant allele showing the replacement of much of Exon 3 by the selectable pMCNeo cassette. The repair contruct for targeting is shown below with the LoxP flanked selectable CMV Hygtk cassette inserted into a SalI site between exons 2 and 3 (CMV, cytomegalovirus promoter; Hygtk, hygromycin resistance/thymidine kinase fusion gene). The next two lines illustrate the structure of the targeted allele and the repaired allele (after Cre recombinase mediated loopout of CMV-Hygtk). Relevant restriction sites and 5′ and internal probes for Southern analysis are shown. Exons are shown as open rectangles (exons 1 and 2 are not to scale). The scale is as shown (kb, kilobase).

FIG. 3 Generation of an ES cell line specifying inducible transgene expression. FIG. 3 shows a schematic representation of integrated expression cassettes. The rtTA is integrated into the constitutive ROSA26 locus on chromosome 6. Cre-mediated recombination of targeting vectors into the homing site on the X chromosome restores resistance to the antibiotic G418 (NEO), thereby facilitating efficient isolation of transgenic cells. TRE, tetracycline response element; PGK, phospoglycerokinase promoter; ATG, methionine initiation codon; lox, recognition sequence for Cre recombinase; GFP, Green Fluorescent Protein; ΔNeo, truncated neomycin (G418) resistance gene.

### DETAILED DESCRIPTION OF THE INVENTION

Somatic Cell Nuclear Transfer

The development of somatic cell nuclear transfer (NT) techniques to produce viable cloned mammals (Wakayama et al., 1998; Wilmut et al., 1997) demonstrated the ability of oocyte cytoplasm to reprogram a somatic donor nucleus to a pluripotent state (Rideout et al., 2001). Additionally, embryonic stem (ES) cells have been derived from blastocysts generated by transfer of somatic cell nuclei (Kawase et al., 2000; Munsie et al., 2000; Wakayama et al., 2001, Hochedlinger, 2002). These “NT ES” cells have been shown to differentiate in vitro into cells of several different developmental lineages, including neurons, blood, and cardiac muscle. In addition, “NT ES” cells were shown to contribute extensively to diploid chimeras (Wakayama et al., 2001) and to generate fertile mice following tetraploid embryo complementation (Hochedlinger and Jaenisch, 2002). Because somatic nuclear transfer allows the isolation of ES cells genetically matched to diseased individuals, this “therapeutic cloning” or “nuclear transplantation therapy” (Vogelstein et al., 2002) approach has been suggested as an attractive possibility to treat a host of medical problems, such as hematopoictic and cardiac disorders, and diseases such as diabetes, Alzheimer's and Parkinson's (Colman and Kind, 2000). In addition, the availability of ES cells opens the prospect for repairing a gene defect by homologous recombination, which has been shown to be effective in correcting a spontaneous mutation in a wild type (wt) ES cell line (Doetschman et al., 1987). Work described herein demonstrates treatment of a genetic disorder through the use of therapeutic cloning combined with gene therapy. In a specific embodiment, therapeutic cloning combined with gene therapy are used to treat (correct) a genetic disorder which is an immune deficiency.

Described herein are results of work that demonstrates the feasibility of correcting a genetic defect in somatic cells of an affected individual using a combination of reprogrammed somatic cell therapy, often designated as “therapeutic cloning” or “nuclear transplantation therapy” (Vogelstein et al., 2002), and gene therapy. The procedure involved the isolation of somatic cells from a mutant mammal in which a genetic defect (mutant genet) resulted in a disorder or condition to be treated. Nuclei from the mutant somatic cells were transferred into enucleated oocytes and ES cells were derived from one of the cloned blastocysts. Such cells are referred to herein as ntES cells. Standard homologous recombination was used to correct the gene defect in the ntES cells, resulting in the production of repaired ntES cells. Assessment of the effectiveness of the repair of the genetic defect showed that it had been corrected and the associated disorder treated.

Results presented herein constitute the first comprehensive proof of principle approach that combines therapeutic cloning with gene and cell therapy to repair a genetic disorder. The methodology described herein can be adapted, using methods described herein and/or methods known to those of skill in the art, to treat (prevent, correct, reverse, or reduce the extent, duration, or severity of) or correct a wide variety of genetic disorders, such as hematopoietic and cardiac disorders, diabetes, Alzheimer's disease, Parkinson's, other neurological conditions and any condition in which the underlying genetic mechanism is known. The present invention can be used to treat conditions in humans and nonhumans (e.g., cows, horses, pigs, goats, dogs, cats, birds). For example, this methodology can be adapted to treat a number of genetic disorders of the hematopoietic system that are currently treated by allogeneic marrow transplantation, including severe forms of hemoglobinopathy (sickle cell anemia, thalassemia) or marrow failure syndromes (Fanconi's anemia) in which the underlying genetic lesion is known. Because ES cells can be differentiated into many therapeutically relevant tissue types including neurons (Lee et al., 2000), cardiac myocytes (Doevendans et al., 2000), and pancreatic beta cells (Lumelsky et al., 2001), the strategy employed here is applicable to a variety of genetic diseases that can be corrected by cell transplantation. Similarly, human ES cells have been shown to be highly proliferative and to differentiate in vitro into cells such as neurons (Reubinoff et al., 2001; Zhang et al., 2001), hematopoietic precursors (Kaufman et al., 2001), and cardiac myocytes (Kehat et al., 2001). “Repaired” human ES cells, which can be selected as precursors of the type of cell desired for use in treating an individual (human or non-human) in need of treatment of a genetic defect (e.g., precursors of neurons, hematopoietic cells, cardiac cells) can be produced, using similar methods, and, optionally, maintained under conditions appropriate for (resulting in) differentiation to produce repaired cells which can be administered to the individual in sufficient dose(s) to result in a therapeutic effect (prevention, correction, reversal or reduction in severity) in the individual. Such repaired human ES cells and cells which result from differentiation of the repaired human ES cells (repaired differentiated human cells, such as repaired HSCs, neurons and cardiac myocytes) are the subject of this invention. Alteration of nucleic acids (DNA, RNA) in the ntES cells, such as repair of a genetic defect which causes or is associated with an undesirable condition or disease to be treated, can be carried out using known methods, such as homologous recombination.

Also the subject of this invention are a method of producing repaired ntES cells, such as repaired mammalian (including human and nonhuman, such as cow, pig, horse, goat, dog, cat and bird) ntES cells and a method of producing repaired differentiated cells derived from or resulting from repaired mammalian ntES (human or nonhuman) cells maintained under conditions that result in their undergoing differentiation. Such cells can be repeatedly expanded as needed and subjected to conditions that result in their differentiation, thus making available a supply of cells for continued therapy.

One embodiment of the method of producing repaired ntES cells comprises introducing somatic cell nuclei obtained from a mutant somatic cell or somatic cell comprising a genetic defect to be repaired into enucleated oocytes; maintaining the resulting oocytes under conditions that result in blastocyst formation, thereby producing blastocysts comprising nuclei/nuclear material from the somatic cells; deriving or obtaining ES cells, referred to as ntES cells, from the blastocysts, wherein the ntES cells comprise the genetic defect present in the mutant somatic cell or somatic cell comprising a genetic defect to be repaired; and correcting the genetic defect in the ntES cells (e.g., by homologous recombination or other method), thereby producing repaired ntES cells. The method of producing differentiated cells, such as differentiated precursor cells (e.g., hematopoietic cells) comprises the steps set forth above and, additionally, maintaining the repaired ntES cells under conditions that result in differentiation of the repaired ntES cells, thereby producing “repaired” precursor cells. The resulting cells can be administered (e.g, intramuscularly, intravenously, or by other route) to an individual in need of treatment of the genetic defect (in need of treatment of the condition or disorder caused by or associated with the genetic defect).

The public debate over therapeutic cloning has emphasized the theoretical potential to derive genetically matched pluripotent cells from the somatic cells of a donor by nuclear transfer into enucleated oocytes. Generating genetically matched pluripotent stem cells for in vitro differentiation into the desired cell type has several potential benefits: (i) no requirement of long term administration of immunosuppressive drugs to prevent rejection of the transplanted cells, (ii) the opportunity to repair genetic defects within stem cells to treat or cure inherited diseases, and (iii) the possibility to repeatedly expand and differentiate the ntES cells into the desired cell type for continued therapy as needed.

Treatment of an Immune Disorder

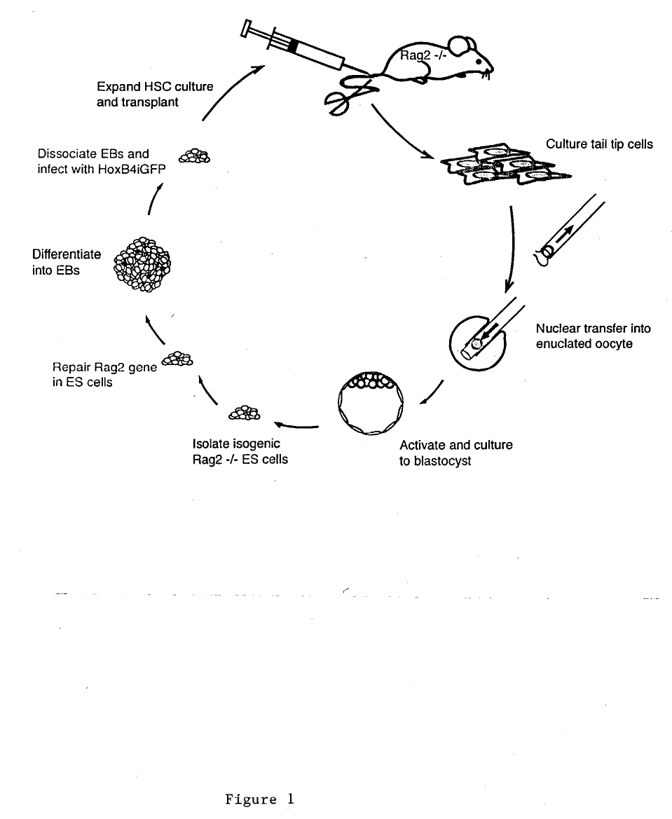

Applicants chose a mouse strain with a defined genetic disorder to develop a model that combines therapeutic cloning with gene and cell therapy (FIG. 1). Tail tip cells from mutant mice that are severly immune deficient due to the mutation of the Rag2 recominases gene. Nuclei from mutant tail tip cells were transferred into enucleated oocytes and ES cells were derived from one of the cloned blastocysts. Standard homologous recombinations was used to creect the gentic defect in the ntES cells. To assess whether the genetic manipulation restored recombinase function, mice were derived from the repaired ntES cells by tetraploid embryo complementation. The lymphoid compartment of the resulting animals consisted entirely of the repaired ES cells and was normal, as demonstrated by B and T cell numbers that are typical for wild type (wt) mice. Rag2 mutant mice that were transplanted with bone marrow from the ntES cell derived mice showed a complete restoration of immune function. Thus, homologous recombination in the ntES cells corrected the genetic defect in the donor Rag2 mutant mouse strain.

The basic steps are as follows: 1) Nuclear transfer of a somatic cell nucleus from the affected donor mouse into an enucleated oocyte, resulting in production of a nuclear transfer (NT) oocyte; 2) activation and cultivation of the NT oocyte to the blastocyst stage; 3) isolation and culture of ES cells (ntES cells) from the blastocyst; 4) repair of the genetic defect in the ntES cells by homologous recombination, resulting in production of repaired ntES cells; 5) differentiation of the repaired ntES cells in vivo, via tetraploid embryo complementation, or in vitro into hematopoietic stem cells (HSCs), resulting in production of repaired HSCs; and 6) transplantation of the “repaired” HSCs into affected donor mice. The model used in the experiments described is the severe combined immune deficiency caused by inactivation of the Rag2 recombinase, which results in the complete absence of mature B and T cells in the lymphoid organs and absence of immunoglobulins from the serum of the mouse (Shinkai et al., 1992). The immune deficiency in Rag1 and 2 knockout mice resembles Omenn syndrome and the severe combined immune deficiency seen in humans homozygous for mutations at either RAG1 or RAG2 (Notarangelo et al., 1999). Rag2 null mice remain viable and have a normal lifespan when housed in a clean animal facility. Importantly, their lymphoid system can be restored by transplantation of isogenic bone marrow or fetal liver hematopoietic stem cells from wild type mice. Therefore, the Rag2 mutant mice provide a sensitive experimental system to detect functional engraftment of hematopoietic stem cells derived from genetically modified ES cells.

Therapeutic cloning for treating an immune deficiency depends on the in vitro differentiation of ntES cells into functional hematopoietic cells that are able to provide long-term repopulation of the lymphoid compartment after transplantation. ES cells can be differentiated in vitro into hematopoietic precursors, as demonstrated by the appearance of blood islands in embryoid bodies (EB) and the isolation of several types of primitive hematopoietic colonies from EBs (Keller et al., 1993; Wiles and Keller, 1991). Recent work showed that expression of the leukemia-associated BCR/ABL oncogene in differentiating ES cells enabled engraftment of mice with leukemic lymphoid and myeloid elements (Perlingeiro et al., 2001). The BCR/ABL experiments also demonstrated that the target cell required for the work described herein the lymphoid-myeloid HSC, was present by day 5 in embryoid bodies derived from ES cells (Perlingeiro et al., 2001). The work described herein made use of a method for deriving normal hematopoietic progenitors by genetic modification of ES cells with the Homeobox gene HoxB4, which provides a means for functional hematopoietic reconstitution of lethally irradiated mice. Results show that “repaired” ES cells derived from a Rag2 deficient mouse can be differentiated into functional hematopoietic stem cells that restore immune function when transplanted into adult Rag2 mutant mice.

Role of the Homeobox Gene HoxB4

A critical step in nuclear transplantation therapy is the derivation in vitro of functional somatic cells from the cloned ES cells that can be used for transplantation into the diseased individual. As described herein, expression of the homeobox gene HoxB4 enables embryonic hematopoietic stem cells to stably engraft and chimerize long-term the lymphoid and myeloid lineages of transplanted mice. The principals described have been applied to generate HSCs from the repaired ntES cells.

Blood development in embryoid bodies (EBs) differentiated from ES cells recapitulates yolk sac hematopoiesis (Keller et al., 1993). Like yolk sac progenitors, ES derivatives are ineffective at repopulating hematopoiesis in lethally irradiated adults, a property believed to reflect defects in homing or responsiveness to the adult bone-marrow microenvironment (Hole et al., 1996; Müller and Dzierzak, 1993; Yoder, 2001). Using the EB differentiation system, it has been shown that primitive progenitors can generate definitive lymphoid, myeloid, and erythroid lineages when engraftment is driven by transformation with the Bcr/Abl oncogene (Perlingeiro et al., 2001). Likewise, yolk sac progenitors can contribute to hematopoiesis in the adult when engrafted into neonates (Yoder et. al, 1997) or when cultured on stroma taken from the para-aortic region of the embryo, where definitive HSCs are first detected (Matsuoka et al., 2001). These data argue that primitive embryonic blood progenitors can be induced to become definitive lymphoid-myeloid hematopoietic stem cells if exposed to the proper microenvironment.

The molecular mechanisms that distinguish primitive and definitive hematopoiesis are largely unknown. Earlier studies have identified several homeotic selector genes that are expressed in definitive HSCs but not in yolk sac, including Hox B3, B4, A4 and A5 (Sauvageau 1994; McGrath and Palis, 1997). HoxB4 was tested as a candidate gene to promote definitive potential for the following reasons: (1) HoxB4 had been shown to enhance hematopoietic repopulation when overexpressed in adult bone marrow, without inducing leukemia or interfering with hematopoictic differentiation (Sauvageau et al., 1995); (2) HoxB4 had been implicated in self-renewal of the definitive HSC (Sauvageau et al., 1995); and (3) HoxB4 had previously been shown to enhance the formation of mixed hematopoietic colonies from differentiating ES cell cultures (Helgason et al., 1996). In this report, we demonstrate that ectopic expression of HoxB4 endows two types of embryonic hematopoictic progenitors (pre-circulation yolk sac and ES-derived progenitors) with the potential to engraft and contribute to multilineage lymphoid-myeloid hematopoiesis in irradiated adult mice.

The present invention is illustrated by the following examples, which are not intended to be limiting in any way.

The following materials and methods were used in Examples 1-5.

NT and ES Cell Derivation

Tail-tip donor cells were cultured for one to two weeks from skinned and macerated 1 cm pieces of 1 month old male mice, Rag2−/− (129B6F1). NT was performed as described (Wakayama et al., 1998). The reconstituted embryos were cultured in mCZB media until they reach the blastocyst stage (generally 4 days) when they were transferred into cultures of mouse embryonic fibroblasts in ES cell media supplemented with 1000U/ml LIF and 50 μg/ml of the MEK1 inhibitor, PD098059. PD098059 has been shown to promote stem cell renewal (Burdon et al., 1999). After 2-3 days in culture most of these tail-tip cell derived blastocysts remained unhatched and were treated with acid tyrode's solution to remove the Zona pelucida. After another 4-5 days in culture, the proliferating ICM was dissociated and placed in a fresh well. After the cell line was passaged, PD098059 was no longer added to the media.

Gene Manipulation Methods

The wild type Rag2 locus was obtained by probing a BAC library (RPCI-22 female 129Sv/EvTAC) with a Rag1 cDNA probe (the Rag1 and 2 loci are closely linked approximately 10 kb apart). The NheI-SpeI fragment (9.3 kb) containing the second and third exons of Rag2 (from BAC clone 390 L-13) was subcloned and a loxP flanked Hygtk selection cassette (ref) was inserted into a unique SalI site in the second intron. This targeting construct had 5′ and 3′ homologous arms of 3.2 and 6.1 kb respectively.

Targeting was carried out as described. Briefly, 50 μg of contruct was linearized (NotI), and electroporated into the ntES cells in HEPES buffered saline (0.4 cm gap cuvette) with a single pulse of 600V, 25 μF. Hygromycin selection (140 μg/ml) was started 24 hours after electroporation. Cre loopout of the selectable marker was done by electroporating 10 μg of pCrePAC plasmid into several targeted ntES cell lines. Gancyclovir (2 μM final concentration) was added 24 hours later.

DNA from ntES cell subclones was isolated as described (Laird et al., 1991). Restriction enzyme digestions were done according to the suppliers guidelines (NEB) on 10 μg of DNA, overnight. Digestions were electrophoresed on 0.85% agarose gels in 0.5×TBE, blotted to nylon membranes (GenescreenPlus) and probed in Church buffer (Church and Gilbert, 1984).

Tetraploid Embryo Complementation

Tetraploid embryo complemenatation was performed as described (Eggan et al., 2001), using B6D2F2 zygotes from C57B1/6 X DBA/2 F1 mice mated together after standard hormone priming. 15-20 ntES cells were injected per tetraploid blastocyst. After transfer into the uterus of pseudopregnant Swiss females (2.5 dpc), Caesarian sections were performed at 19.5 dpc and live pups were fostered.

Mouse Strains

The Rag2−/− mice were F1s of 129Sv/EvTac X C57B1/6. Rag2−/−, gamma C−/− mice were a mixed background of C57B1/6 and C57B1/10.

ES Cell Propagation and Differentiation

ES cells were grown on primary irradiated mouse embryonic fibroblasts in standard ES cell media, high glucose DMEM (Gibco/BRL) containing 15% fetal calf serum (Hyclone), 1× penicillan/streptomycin, 1× non essential amino acids (Gibco/BRL), 4 μl/500 mls betamercaptoethanol, and 1000 U/ml LIF. They were induced to differentiate into hematopoietic progenitors as described herein. Day 6 EBs were disrupted with collagenase and plated into 6-well dishes of semi-confluent OP9 stromal cells at 105 cells per well for retroviral infection with MSCVHoxB4iGFP as described herein. The MSCVHoxB4iGFP retrovirus was the best of several constructs in inducing the primitive to definitive hematopoietic transition in yolk sac derived cells. Colonies that arose were expanded by transferring each well's contents (adherant and nonadherent cells) by trypsinization onto a T175 flask with semiconfluent OP9 cells. Differentiated ntES cells were used for transplantation 14 days after retroviral infection.

Transplantation Procedures

Rag2−/− mice receiving neonatal blood or bone marrow grafts were given a single dose of 450 Rads, prior to lateral tail vein injection of neonatal blood or bone marrow from ntES derived mice from tetraploid embryo complementation. Rag2−/−, gamma C−/− mice were given 950 Rads fractionated into two doses seperated by 4 hours. 2×10⁶ differentiated ntES cells in IMDM/10% IFS were injected into each animal.

FACS Sorting and ELISA

Peripheral blood lymphocytes (PBLs) and splenocytes were treated with ACK lysing buffer (0.15 mM NH₄Cl, 10 mM KHCO₃, 0.1 mM Na₂EDTA, pH 7.2) prior to FACS analysis to remove red blood cells. 1×10⁶ cells were stained with PE-B220 and FITC-IgM or PE-IgM antibodies to detect B cells, FITC-CD4 or PE-CD4, and PE-CD8 antibodies to detect T cells. Propidium iodide was added to exclude dead cells. All antibodies were purchased from Pharmingen. FACS analyses were performed on a Becton-Dickinson cell sorter.

ELISA was done using the clontyping kit from Southern Biotechnology according to manufacturer's specifications.

PCR Analysis

Primers for detecting the Rag2+^{R} allele [(KH1, TGCGAAGGGACTAGATGGAC (SEQ ID NO.: 11); KH2, CAACCATACGGGCTAGAAGC (SEQ ID NO.: 12)] were designed by the Primer 3 program (Rozen and Skaletsky, 1998) and amplifications were performed on 50 ng of sample DNA in standard PCR conditions for Taq (Gibco) for 34 cycles of 95° C., 30 sec; 60° C., 30 sec, 72° C., 30 sec, followed by 70° C. for 5 min. The residual sequences left behind after Cre mediated loopout of the selectable marker result in a 400 bp product for the repaired allele, while wt and mutant Rag2 alleles give a 200 bp band.

Primers for TCRβ rearrangements were as described (Whitehurst et al., 1999) using primer pairs 1 and 4, 1 and 7, or 5 and 7. Primers for PCR of IgH rearrangments (V to DJ) were as described (Schlissel et al., 1991); a mixture of three degenerate oligonucleotides (V_{H}7183, V_{H}558, and V_{H}Q52 and the J3 primer. TCRβ and IgH PCRs were performed in standard Taq conditions (Gibco) for 35 cycles of 95° C., 1 min; 62° C., 1 min; 72° C. for 2.5 min. All PCR products were anaylzed by gel electrophoresis in 1.5% agarose, 0.5×TBE and stained with ethidium bromide.

### EXAMPLE 1

### NT and Pluripotent ntES Cell Derivation

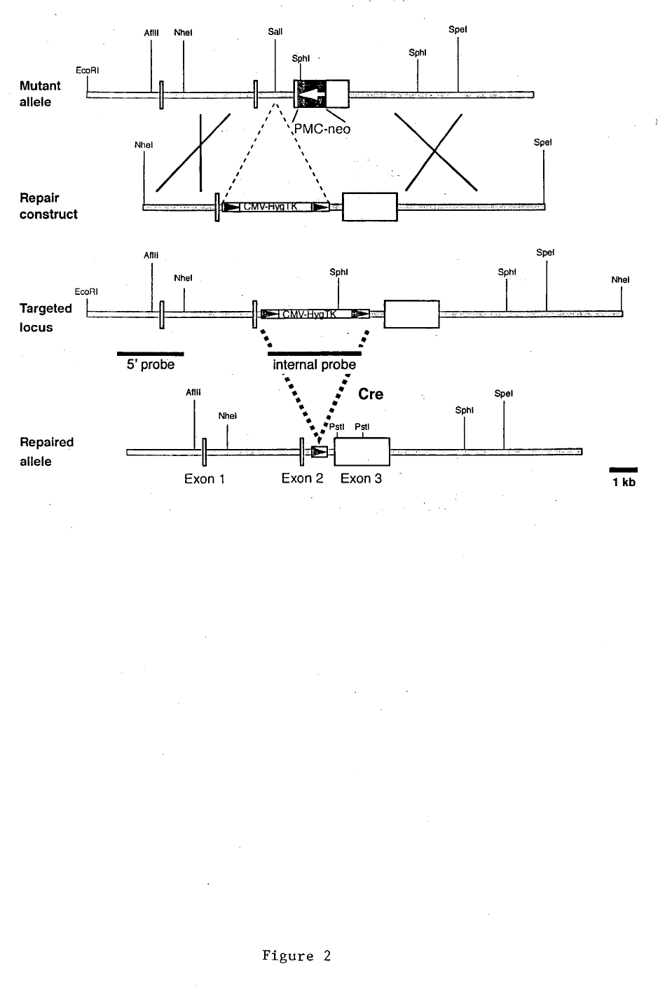

The immuno-deficient mouse model for Rag2 deficiency has previously been generated by deletion of part of the third coding exon and the insertion of a pMCneo cassette transcribed in the opposite orientation (FIG. 2) (Shinkai et al., 1992). Tail-tip cells from a Rag2−/− male mouse (129Sv/Ev X C57B1/6 (129B6F1)) were used as nuclear donors for transfer into enucleated MII (metaphase II) oocytes by the “Honolulu” method (Wakayama et al., 1998). The development of the tail-tip NT embryos to the blastocyst stage was usually delayed compared to in vitro activated and cultured parthenogenetic embryos (4.5 and 3.5 dpc, respectively). Approximately 13 percent (27 of 202) of the reconstructed oocytes developed into blastocysts and of these one generated an ES cell line (Rag2−/− ntES). While this rate of blastocyst formation following nuclear transfer from tail-tip cells was lower than that reported by others (13% vs. 38%) (Wakayama et al., 2001), the rate of ES cell derivation from the cloned blastocysts was comparable (3% vs. 6%).

The resulting cell line, Rag2−/− ntES, was tested for pluripotency by the most stringent method available, namely, tetraploid embryo complementation (Eggan et al., 2001; Hochedlinger and Jaenisch, 2002; Nagy et al., 1993). ES cell complementation of tetraploid host blastocysts results in the embryo being completely derived from the injected ES cells while the tetraploid host cells contribute to the placenta (Wang et al., 1997). Injection of wild type Fl ES cells into tetraploid blastocysts has previously been shown to result in viable mice from 4-10% of the manipulated embryos (Eggan et al., 2001). Injection of ntES cells derived from F1 lymphocytes also gave rise to viable mice (Hochedlinger and Jaenisch, 2002). Similarly, the Rag2−/− ntES line generated 4 viable pups out of 14 reconstituted tetraploid blastocysts (28%) indicating that this line was pluripotent and able to efficiently generate all somatic cell types (Table 1).

### EXAMPLE 2

### Repair of the Rag2 Mutation in the Rag2−/− ntES Line

Rag2 function in the Rag2−/− ntES line was restored by homologous recombination, followed by cre recombinase mediated removal of the loxP flanked selectable marker (Hygtk) (FIG. 2). Because the selectable marker was positioned close to the site of the insertion/deletion mutation of the Rag2 null allele (approximately 0.5 kb), recombination occurring between the site of the Hygtk cassette and pMC-Neo insertion in the mutant allele was unlikely. Southern analysis of DNA from Hyg resistant subclones was performed with a 5′ probe to check for homologous recombination and an internal probe to exclude random integrations (data not shown). Correct targeting was found in 58/288 (20%) of the subclones demonstrating that NT derived ES cells can be effectively targeted by homologous recombination like normal mouse ES cells.

Two targeted subclones (#4 and #132) were transiently transfected with a cre expressing plasmid (pCrePAC) (Taniguchi et al., 1998) and selected with gancyclovir for loop-out of the Hygtk selectable marker (FIG. 2). Southern analysis with the 5′ probe on DNA from gancyclovir resistant subclones detected the loss of the selectable Hygtk marker and no additional gene rearrangements in the repaired allele. This restored normal Rag2 gene structure on one allele and left a single loxP site in the second intron (this allele was designated, Rag2+^{R}) In order to assess whether the gene targeting had restored proper gene function, mice were generated from the repaired ES cells.

### EXAMPLE 3

### Tetraploid Embryo Complementation with the Rag2+^{R}/− ES Lines

The genetically repaired ntES cells were used to generate mice by tetraploid embryo complementation (Hochedlinger and Jaenisch, 2002), in which the embryo proper is entirely derived from the ntES cells and the extraembryonic lineages are derived from the tetraploid host blastocyst. Therefore, successful correction of the Rag2 mutation can be directly assessed by analyzing immune function in the “repaired” ES cell derived animals. Furthermore, neonate blood (analogous to cord blood transplants) or adult bone marrow harvested from the animals can be transplanted into adult Rag2 mutant recipients to evaluate their potential to colonize the lymphoid compartment and correct the immune deficiency. 226 tetraploid blastocysts were injected with 4 different repaired subclones (4-4, 132-1, 132-2, and 132-3); 38 live pups (16%) were delivered by C-section (Table 1). Of these, 9 died shortly after delivery, but the rest were viable, healthy, and fertile. Thus, the repaired Rag2+^{R}/− ntES cells remained fully pluripotent, with no loss of developmental potential.

The lymphoid cells of the Rag2+^{R}/− ntES mice derived by tetraploid embryo complementation were analyzed to determine whether the repaired allele was functional. PCR analysis to detect rearrangements at the immunoglobulin heavy chain and T-cell receptor b loci showed the presence of multiple rearranged alleles in the thymus and spleen of Rag2+^{R}/− ntES derived mice, while no rearranged alleles and only the germline allele were seen in mice derived from the original Rag2−/− ntES line. In addition, peripheral blood from Rag2−/− ntES and Rag2+^{R}/− ntES derived mice was compared to blood from a wild type mouse by flourescence activated cell sorting (FACS) with antibodies against markers for B cells (B220 and IgM) and T cells (CD4 and CD8). The relative numbers of B and T cells detected in Rag2+^{R}/− ntES mice were comparable to the B and T cell populations in wild type mice; in contrast, blood from mice derived from the parental Rag2−/− ntES line showed essentially no mature B and T cells. This proved that the repaired Rag2 allele could restore normal TCR and immunoglobulin rearrangements, and enable B and T cell production during normal development of mice derived by tetraploid embryo complementation.

The mice derived from the repaired ES cells were used as HSC donors (from neonate peripheral blood and bone marrow from 1 month old mice) for transplantation back into sublethally irradiated Rag2 null mice. After two to three months the mice were bled and analyzed by FACS, which showed the presence of mature B and T cells. The relative level of mature B and T cells (30.3%±19.2 and 27.7%±6.1 (n=6), respectively) in total peripheral blood mononuclear cells (PBMCS) was similar to that of normal mice. This indicates that the repaired ntES cells gave rise to normal bone marrow that restored the lymphoid system after transplantation into Rag2 mutant mice. The donor HSCs in these experiments were generated during the course of normal mouse development in the tetraploid complementation embryos. The restoration of immune function in the recipients indicated that bone marrow cells derived from the “repaired” ES cell mice were able to fully function after transplantation into Rag2 mutant host animals.

Following is a description of assessment of whether in vitro differentiation of the repaired ES cells, rather than in vivo formation of normal bone marrow, would allow the generation of definitive hematopoietic stem cells that could be used for transplantation into mutant animals.

### EXAMPLE 4

### In vitro Differentiation of Rag2+^{R}/− ntES Cells and Transplantation into Rag2 Mutant Mice

Therapeutic cloning requires that the ntES cells be differentiated in vitro into the relevant tissue or cell types followed by transplantation into affected nuclear donors. Described herein is a method for in vitro differentiation of ES cells into embryonic hematopoietic stem cells that could be used for long term lymphoid and myeloid engraftment of lethally irradiated. This system was used to attempt restoration of immune function in immunodeficient Rag2−/− mice with the Rag2+^{R}/− ntES cells. The Rag2+^{R}/− ntES cells were differentiated into EBs for 6 days. Subsequently the HoxB4 and GFP (green fluorescent protein) genes were introduced into the cells by retroviral transduction with the MSCVHoxB41GFP vector. The infected cells were then cultured for 14 days on OP9 stromal cells in the presence of hematopoietic cytokines to promote formation of HSCs that could be used for transplantation into Rag2−/− isogenic mice. The initial transplantation of hematopoietic derivatives of Rag2+^{R}/− ntES cells into Rag2−/− mice showed little to no chimerism of the hematopoietic compartment as assessed by the numbers of GFP positive cells in the peripheral blood of recipient mice. This suggested that resistance to engraftment was a property of the Rag2-deficient recipients and not the ntES cells, because HoxB4-modified embryonic hematopoietic stem cells engrafted in isogenic wild type but not Rag2-deficient recipients.

### EXAMPLE 5

### Host NK Cells Present a Barrier to Engraftment of Hematopoietic Progeny of the ntES Cells

Yolk sac hematopoietic progenitors, which are closely related to EB-derived progenitors, have lower major histocompatability complex (MHC) expression than bone marrow derived HSCs (Cumano et al., 2001; Huang and Auerbach, 1993) and it is known that hematopoictic cells with low MHC expression are a target for NK cell mediated rejection (Bix et al., 1991). Indeed, expression of the two class I MHC genes (H2-K^{B} and H2-D^{B}) was significantly lower in the Rag2+^{R}/− ntES derived HSCs than in bone marrow. Therefore, it appeared possible that enhanced NK activity in Rag2 mutant recipients was preventing engraftment of ES cell derived HSCs.

This hypothesis was tested using two different approaches. First, pretreatment of the Rag2−/− mice with an anti-NK1.1 antibody which depletes NK cells responsible for the phenomenon of hybrid resistance prior to transplantation (Kung and Miller, 1995; Lee et al., 1996), resulted in low level reconstitution of hematopoietic cells in the peripheral blood as assessed by FACS. A small minority of these cells stained with B220 antibody (0.13% of total PBMC) demonstrating that the repaired ntES cells contributed to the B cell lineage. However, staining for IgM positive B cells and mature CD4 and CD8 positive T cells was essentially negative. Analysis of these mice showed persistence of NK1. 1 positive cells in peripheral blood, suggesting that immunodepletion was inefficient and incomplete. Therefore, in the second approach, hematopoietic derivatives of the Rag2+^{R}/− ntES cells were engrafted into Rag2−/− recipients with a complete absence of NK cells due to deletion of the IL2 common cytokine receptor gamma chain (gamma C) (Mazurier et al., 1999). This strategy has recently been shown to enhance engaftment of definitive intraembryonic populations of hematopoietic progenitors (Cumano et al., 2001). FACS analysis of PBMCs from these double mutant animals transplanted with the Rag2+^{R}/− ntES cells showed essentially complete donor chimerism, with predominantly myeloid repopulation as shown by extensive staining with GR1, a marker for granulocytes, and limited staining with B220, a marker for B cells. FACS analysis detected a low level of GFP-positive, mature B cells by IgM staining (0.74%) and GFP-positive, mature T cells by CD4 and CD8 staining (0.09% and 0.38%, respectively). The detection of lymphocytes in the peripheral blood suggested that some lymphoid progenitors derived from the ntES cells were maturing in the engrafted mice.

To ensure that the engrafted cells in the double mutant mice were derived from the repaired ntES cells, PCR analysis was preformed to detect the Rag 2+^{R} allele. The repaired allele was detected in DNA isolated from hematopoietic tissues of mice transplanted with in vitro differentiated ES cells or neonate blood/bone marrow from ntES derived mice by tetraploid embryo complementation. In contrast, the repaired allele was absent in DNAs from wt and Rag2−/− control animals.

To confirm that the proper rearrangements necessary for B and T cell function had occurred in transplanted double mutant mice, PCR analyses of IgH and TCR loci were performed on lymphoid organs of a mouse 3.5 weeks after transplantation. Multiple rearranged alleles were detected, indicating that the transplanted ntES cell derivatives gave rise to polyclonal reconstitution of the B and T cell compartments. The level of TCR b gene rearrangement was about 20% of that seen in Rag2+^{R}/− ntES mice, derived by tetraploid embryo complementation, or wt mice. Levels of detectable IgH gene rearrangement in the spleen was much lower, approximately 2% of wt. Moreover, Applicants tested serum of ntES cell engrafted animals and controls for the presence of immunoglobulins of the IgM, IgG, and IgA classes. In contrast to the untreated Rag2−/−, gamma C−/− control, all treated mice demonstrated the presence of serum IgM, IgG, and IgA. In agreement with the fewer peripheral blood lymphocytes in the ntES treated mice, serum Ig levels, particularly IgA, were lower than in controls. IgM levels were 10-15 fold lower in the engrafted mice compared to wt and IgG and IgA were approximately 125 fold lower. Thus, despite low levels of B and T cells in the peripheral blood of the Rag2−/−, gamma C−/− mice, some immune function was restored in the mice engrafted with in vitro repaired and differentiated ntES.

As discussed below, there were two challenges in treating the immunodeficiency in the model described. First, attempts at hematopoietic repopulation were hindered by an engraftment barrier peculiar to the Rag2-deficient recipients, which Applicants have linked to NK cell function. Second, the repaired cells preferentially engraft the myeloid lineages and show a relative block to T cell maturation by an as yet undefined mechanism. Therefore, while initial attempts at therapeutic cloning have succeeded in restoring a modest degree of immune function, the present work uncovered interesting and unanticipated biological principles that must be more fully defined to make therapeutic cloning more successful in this system. Applicants' current state of understanding of these challenges is outlined below.

The ntES cell derived HSCs express low levels of MHC. Because it has been well established that low MHC expression on HSCs can lead to NK cell mediated graft rejection (Bix et al., 1991), and that Rag2-deficient mice retain NK cell function, Applicants tested whether inhibition of NK activity would improve engraftment of ES donor cell derived HSCs into Rag 2 recipients. Initial pilot experiments with immunodepletion of NK cells in the Rag2−/− mice prior to transplantation resulted in low level engraftment of the in vitro derived HSCs (1.5% chimerism in PBMCs). In contrast, engraftment was essentially complete (95% peripheral blood chimerism) in a Rag2 null strain rendered devoid of NK cells by virtue of genetic deletion in the IL2 common cytokine receptor gamma chain (gamma C) knockout. Results raise the provocative possibility that even genetically matched cells derived by therapeutic cloning may still face barriers to effective transplantation for some disorders.

Despite high level chimerism in the reconstituted Rag2−/−, gamma C−/− double mutant mice, Applicants observed a predomincance of myeloid cells and a pancity of lymphoid cells in the peripheral blood. Analysis of lymphoid organs has shown extensive chimerism of the thymus and spleen and evidence of TCR and IgH gene rearrangement respectively, suggesting a blockade to release of the lymphoid populations into the peripheral circulation. For several reasons, Applicants believe that this relative block to lymphoid differentiation is due to the retroviral mediated constitutive expression of HoxB4 and not to any specific defect in the repaired ntES cells. The capacity for mature lymphoid development from the repaired ntES cells is clear from the observation of functional lymphoid reconstitution in the animals derived from tetraploid embryo complementation. Furthermore, it has been shown previously that a fully functional lymphoid system can be reconstituted, albeit transiently, from in vitro differentiated ES cells (Potocnik et al., 1997).

Though original reports employing retroviral transduction of murine bone marrow with a HoxB4 retrovirus showed no disruption in hematopoiesis (Sauvageau et al., 1995), more recent data suggests that high level expression of HoxB4 by adenoviral transduction enhances myeloid differentiation in a concentration-dependent manner (Brun et al., 2001), and retroviral expression of the related HoxB3 protein has been linked directly to inhibition of lymphoid differentiation (Sauvageau et al., 1997). These reports corroborate Applicants' experience that shows the retroviral expression of HoxB4 in the in vitro culture system to yield less consistent lymphoid reconstitution than the inducible expression system. High level constitutive expression of HoxB4 may, therefore, drive hematopoietic engraftment but skew differentiation away from the very lymphoid populations Applicants were attempting to restore. Though enough maturation of lymphocytes occurs to reconstitute some level of immunoglobulin in serum, the immune reconstitution is incomplete. Overcoming this problem might require engineering the inducible system for HoxB4 expression into the Rag2+^{R}/− ntES cells or devising a differentiation protocol not dependent on HoxB4.

The ability to derive pluripotent cells by NT is not limited to a single species (Cibelli et al., 1998). Derivation of human NT EScells might be possible. It is of interest, that while the efficiency of deriving ES cells from NT embryos is low (approximately 2.2% from tail tip cells (Wakayama et al., 2001)), it appears to be greater than the efficiency of obtaining viable clones from NT embryos (0.5% from mouse tail-tip cells (Wakayama et al., 1999)). The more efficient derivation of ntES cells than of viable animals may result from the in vitro expansion of a few successfully reprogrammed cells in an otherwise failing blastocyst. The ntES cells derived from somatic cells have regained complete developmental potential (pluripotency), as evidenced by the ability to derive mice through tetraploid embryo complementation (Hochedlinger and Jaenisch, 2002). The pluripotency of the ntES cells did not appear to be impacted by the genetic manipulation and substantial time in tissue culture required to execute their genetic repair. Thus, murine ES cells derived from “therapeutic cloning” are highly proliferative and as facile to genetic manipulation as wt ES cells, making them an integral tool in studying cell replacement based gene therapies.

The following materials and methods were used in Examples 6-9. Cell culture: ES cells were maintained on irradiated MEFs in DME/15% IFS, 0.1 mM non-essential amino acids (Gibco), 2 mM glutamine, penicillin/streptomycin (Gibco), 0.1 mM β-mercaptoethanol, and 1000 U/mL LIF (Peprotech). For EB differentiation, ES cells were trypsinized, collected in EBD (IMDM/15% IFS, 200 μg/mL iron-saturated transferrin (Sigma), 4.5 mM monothiolglycerol (Sigma), 50 μg/mL ascorbic acid (Sigma), and 2 mM glutamine) and plated for 45 minutes to allow MEFs to adhere. Nonadherent cells were collected and plated in hanging drops at 100 cells per 10 μL drop in an inverted bacterial petri dish. EBs were collected from the hanging drops at day 2 and transferred into 10 mL EBD in slowly rotating 10 cm petri dishes. At day 4, EBs were fed by exchanging half of their spent medium for fresh EBD. Cells were harvested at day 6 by collagenase treatment. Retroviral supernatants were produced in 293 cells by FUGENE co-transfection, according to the manufacturer's specifications, of viral plasmid with packaging-defective helper plasmid, pCL-Eco (Naviaux et al., 1996). 293 cells were grown in DME/10% inactivated fetal calf serum (IFS), and medium was replaced the day after transfection. 10⁵ EB or 10⁴ yolk sac cells were resuspended in 3 mL of retroviral supernatant with 4 μg/mL polybrene and cytokines (100 ng/mL SCF, 40 ng/mL VEGF, 100 ng/mL TPO, 100 ng/mL Flt-3 ligand), transferred to semiconfluent OP9 cells in 6-well dishes, and centrifuged at 2500 rpm for 90 minutes at 33° C. After spin-infection cells were returned to 37° C. for overnight incubation and the next morning the medium was exchanged for IMDM/10% IFS and the same cytokines. When confluent, the cultures were passaged by pooling suspension and semi-adherent cells (obtained by trypsinization) and replating on to fresh OP9 or injecting into adult recipients. Colony assays were done in methylcellulose medium with IL3, IL6, Epo and SCF (M3434, StemCell Tech.).

Generation of MSCV-HoxB4iresGFP retrovirus: The HoxB4 cDNA was subcloned as an Eco RI-Xho I fragment from MSCV-HoxB4-Puro (Helgason et al., 1996) into MSCViresGFP (Van Parijs et al., 1999).

Generation of the lox targeting plasmid: The lox-targeting plasmid, plox, was generated by subcloning the Sal I (blunted)-Hind III fragment of pPGK-loxP-Xist (Wutz et al., 2002) into Bgl II (blunted)-Hind III cut pNeoEGFP (Clontech). plox has a stuffer fragment (the EGFP gene) derived from pNeoEGFP, bounded by multiple cloning sites upstream and downstream. We replaced the stuffer by digesting with Eco RI and Sal I and inserting the HoxB4 cDNA from MSCV-HoxB4iresGFP on an Eco RI-XhoI fragment to generate ploxHoxB4.

Generation of the doxycycline-inducible HPRT target ES cell line, and the inducible HoxB4 cell line: The Sal I-Mlu I fragment from pHPRT-pBI-EGFP-loxNEO (Wutz et al., 2002) was subcloned into Sal/Mlu cut pneoEGFP (Clontech) in order to place an Xho I site downstream of Mlu I. The resulting Sal I-Xho I fragment was subcloned into Sal I cut pBluescript in order to place a Not I site downstream of Mlu I. Digestion of this plasmid with Not I liberated a fragment containing the tet response element and the loxΔNEO gene. This fragment was ligated into the Not I site of the HPRT targeting vector (Bronson et al., 1996). Two orientations are possible: we selected the orientation in which the lox site is in between the HPRT upstream sequence and the ΔNEO gene, the opposite orientation as was used by Wutz et al. The resulting plasmid was linearized with Sal I and then electroporated into E14-nlsrtTA-7 ES cells (Wutz et al., 2002). After 10 days of selection in ES medium with HAT (Sigma), colonies were picked, expanded, and proper integration was confirmed by Southern blotting. This cell line, named Ainv 15 was targeted with ploxHoxB4 by coelectroporation of 20 μg each of ploxHoxB4 and the Cre recombinase expression plasmid, pSalk-Cre (generously provided by Stephen O'Gorman) followed by selection in ES medium with 300 μg/mL G418 (Gibco) and isolation of clones to generate the inducible cell line, iHoxB4. Protein extracts from iHoxB4 ES cells were tested by Western blotting using the 112 anti-HoxB4 monoclonal antibody (Gould et al., 1997). Blots were probed with a 1:50 dilution of hybridoma supernatent in PBS/5% skim milk powder/0.05% Tween-20, and visualized with HRP-conjugated goat-anti-rat secondary antibody (Santa Cruz Biotechnology, sc2006).

Yolk sac isolation: Pregnant female 129SvEv mice (Taconic) were sacrificed 8.25 days post copulation (the appearance of a vaginal plug was taken as day 0.5). Yolk sacs were separated from the embryo proper (which were examined to exclude yolk sacs from embryos with 5 or more somite pairs) and disaggregated by collagenase treatment.

Transplantation: 2-3 month old 129SvEv females (isogenic to the yolk sac cells) and 129Ola/Hsd (Harlan; isogenic to the ES cells) were given 2×500 cGy doses of gamma irradiation, separated by 4 hours, and injected with 2×10⁶ cells in 500 μL IMDM/10% IFS via lateral tail vein.

RTPCR: Primers: actin(f) 5′-GTGGGGCGCCCCAGGCACCA-3′

actin(r) 5′-CTCCTTAATGTCACGCACGATTTC-3′

β-H1(f) 5′-AGTCCCCATGGAGTCAAAGA-3′

β-H1(r) 5′-CTCAAGGAGACCTTTGCTCA-3′

β-maj(r) 5′-CTGACAGATGCTCTCTTGGG-3′

β-maj(r) 5′-CACAACCCCAGAAACAGACA-3′

CXCR4(f) 5′-TCAAGCAAGGATGTGACTTCGA-3′

CXCR4(r) 5′-AGGTCCTGCCTAGACGCTCATT-3′

TEL(f) 5′-CTGAAGCAGAGGAAATCTCGAATG-3′

TEL(r) 5′-GGCAGGCAGTGATTATTCTCGA-3′

The above sequences are, respectively, SEQ ID NOS.: 1-10.

Cycle Conditions: 2 min. at 94° C.; 30 cycles of (45 sec. At 95° C.; 1 min. at 60° C.; 1 min at 72° C.); 5 min at 72° C.

### Example 6

### HoxB4 Transduction of Yolk Sac Cells

The HoxB4 cDNA was expressed in cells isolated from pre-circulation murine yolk sac (E8.25, 2-4 somite pair embryos) using a retrovirus that co-expressed GFP (Van Parijs et al., 1999). Cells were grown on an OP9 stromal cell layer, previously shown to support maintenance of hematopoietic progenitors derived from ES cells in vitro (Nakano et al., 1994). HoxB4-infected cultures gave rise to abundant colonies of semi-adherent cells with hematopoietic blast morphology, while control cultures showed no growth. Cultured cells were injected into four lethally irradiated syngeneic adult recipients, which were assayed over time for GFP-positive cells in the peripheral blood. Bone marrow from one primary mouse was examined for donor-derived GFP-positive cells counter-stained with antibodies specific for myeloid and lymphoid hematopoietic lineages. Recipients showed donor-derived engraftment of myeloid (Gr-1+ and Mac-1+), B lymphoid (B220+) and T lymphoid (CD4+ and CD8+) cells, demonstrating that HoxB4 expression confers on pre-circulation yolk sac cells the capacity for engraftment and multi-lineage differentiation in irradiated adults. Donor-derived bone marrow cells from primary animals were transplanted into secondary recipients, where they contributed to multilineage hematopoiesis for at least five months, the longest time point analyzed in this study. However, lymphoid engraftment waned over time in secondary animals, an observation Applicants have linked to the inhibitory effects of constitutive HoxB4 expression on lymphoid differentiation, as discussed below. The data demonstrate that HoxB4 expression induces definitive hematopoietic stem cell potential in primitive yolk sac-derived hematopoietic precursors isolated prior to the onset of circulation.

### EXAMPLE 7

### HoxB4 Induction in Embryoid Body-Derived Cells

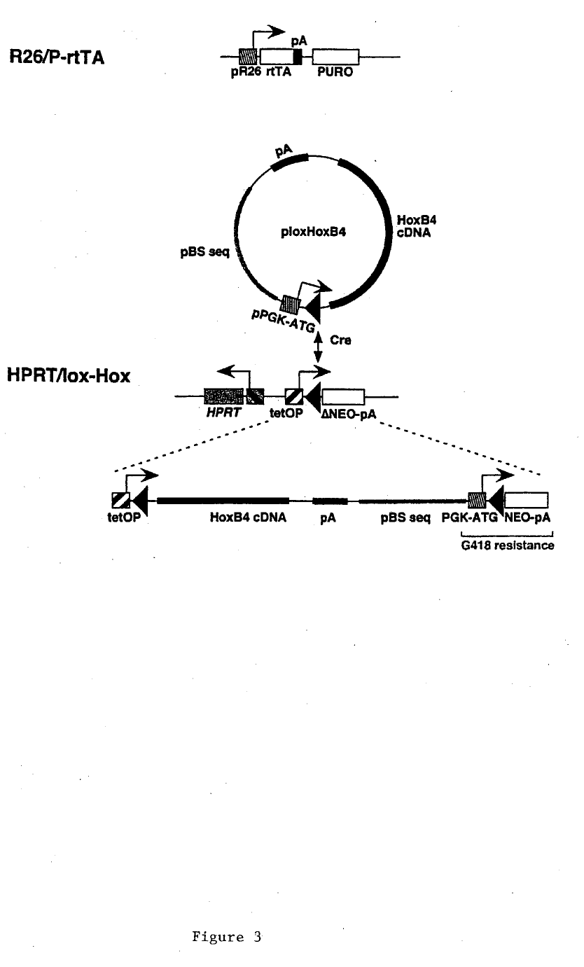

The same retroviral construct was used to infect cells from day 6 EBs and Applicants found that HoxB4 expression produced a similar outgrowth of hematopoietic blast cells on OP9 stroma (not shown). However, the results with yolk sac cells suggested that constitutive retroviral expression might have undesirable effects on hematopoietic differentiation. To achieve more consistent and homogenous induction of HoxB4, and to enable reversible HoxB4 expression in vitro and in engrafted animals, Applicants generated a tetracycline-inducible HoxB4 transgene in ES cells. First, the reverse tetracycline transactivator (rtTA; Gossen et al., 1995) was inserted by homologous recombination into the constitutively active ROSA26 locus (Zambrowicz et al., 1997). Then, a targeting was introduced site upstream of the HPRT locus such that site-specific integration of transgene constructs would regenerate a functional antibiotic resistance gene (NEO), thereby facilitating efficient selection of transgenic cells (FIG. 3; Wutz et al., 2002).

FACS analysis of a transgenic ES cell line targeted with a GFP reporter construct demonstrated no detectable reporter expression in uninduced ES cells, thereby confirming the low basal rate of the conditional promoter. Following induction with the tetracycline analogue doxycycline, GFP was readily detected in undifferentiated cultures of ES cells. Robust expression was also seen when induction was started at day 8 of EB differentiation, and maintained for 48 hours. Thus the GFP reporter was free from the transgene silencing frequently seen in differentiated ES cells. HoxB4 was targeted into the inducible locus, and expression of HoxB4 protein was assessed by western blotting with a monoclonal antibody to HoxB4 (Gould et al., 1997). Expression was detectable only in doxycycline-induced ES cells.

The effect of HoxB4 induction on hematopoiesis was tested by exposing EBs to doxycycline from day 4 to day 6 of differentiation, the time at which the hemangioblast undergoes commitment to the primitive HSC (Perlingeiro et al., 2001). At day 6 the EBs were dissociated and plated in methylcellulose suspension culture to score for hematopoietic colony forming cells. HoxB4 induction had a marked stimulatory effect on the most immature multipotential myeloid progenitor detectable in this assay, the CFU-GEMM. CFU-GEMM from uninduced EBs were sparse with a relatively limited erythroid burst, whereas HoxB4 induction generated larger, denser colonies that resembled CFU-GEMM from bone marrow. Applicants cultured cells from the day 6 EBs on OP9 stroma in media supplemented with cytokines and doxycycline to maintain HoxB4 expression. This yielded colonies of semi-adherent cells with hematopoietic blast-like morphology that closely resembled the HoxB4-transduced yolk sac cells grown under comparable liquid culture conditions. The expanded cells generated definitive myeloid colony types in methylcellulose media. We characterized the cultured cells for surface antigen expression by FACS and found that the majority expressed the HSC markers c-kit and CD31 (table 2). In addition, Applicants noted minor populations of cells that expressed differentiation markers of the myeloid (Mac-1 and Gr-1), and to a lesser extent erythroid (Ter119) and lymphoid (B220) lineages. Thus, the cultured cells consist of immature hematopoietic progenitors undergoing substantial self-renewal and modest differentiation in culture.

### Example 8

### Markers of Definitive Hematopoiesis in HoxB4-Modified Progenitors

Experiments were carried out to determine whether HoxB4 expression in cultured primitive yolk sac and ES-derived progenitors might induce expression of genes linked to the primitive-definitive transition. By RT-PCR the yolk sac from day 8.25 embryos expressed both embryonic β-H1 and adult-type β-major globins. In contrast, HoxB4-modified yolk sac and ES-derived populations all showed silencing of β-H1 globin in favor of expression of P-major, suggesting that HoxB4-expression and growth on OP9 stroma extinguished primitive erythroid potential. The small amount of β-H1 seen in the retrovirally transduced EB sample (EB:rv-HoxB4) is likely due to contamination with uninfected cells. Applicants also examined two genes important for homing of the definitive hematopoietic stem cell to the adult bone marrow: CXCR4, required for stem cell homing after transplantation (Peled et al., 1999), and TEL, which plays a critical role in the transition of hematopoiesis from the fetal liver to the bone marrow (Wang et al., 1998). Neither CXCR4 nor TEL were detectable in yolk sac, but both were expressed in HoxB4-modified yolk sac and ES-derived hematopoietic populations. Applicants conclude that HoxB4-expression, combined with expansion on OP9 stroma, confers markers of definitive hematopoiesis on these cells of primitive embryonic origin.

### EXAMPLE 9

### Engraftment of ES-Derived Hematopoietic Progenitors in Irradiated Mice

HoxB4-induced ES-derived hematopoietic cells were transplanted into irradiated syngeneic mice in order to assay their ability to engraft and differentiate in the adult environment. For this purpose, cells were first labeled by infection with the MSCViresGFP retrovirus and FACS sorted for GFP+ cells before intravenous injection. Using GFP expression as a marker Applicants found that 5-32% of bone marrow mononuclear cells were donor derived two weeks post-transplant, demonstrating that injected cells could home to the bone marrow. At twelve weeks post-transplant, substantial contributions to myeloid and lymphoid lineages were detected by simultaneous two-color detection of GFP and differentiation markers for myeloid (Gr-1, Mac-1), B-lymphoid (B220) and T lymphoid (CD4, CD8) lineages. Applicants monitored GFP+donor cells over time in engrafted mice by serial sampling of peripheral blood. Despite exposure to 1000 cGy of gamma irradiation, all animals showed mixed chimerism with donor and host-derived cells. Maintenance of HoxB4 induction in vivo was not necessary for sustained donor engraftment, suggesting that HoxB4 expression during in vitro culture was sufficient to confer definitive potential. Applicants detected donor-derived GFP+ cells expressing the HSC markers c-kit, Sca-1, and AA4 in the bone marrow of engrafted mice, suggesting that the transplanted cells were represented in the hematopoietic stem cell pool.

To assess whether long term repopulating HSCs were generated, Applicants transplanted donor-derived bone marrow cells from engrafted primary mice into secondary recipients, and detected donor cells in secondary mice over five months. The donor and secondary recipients were not exposed to doxycycline, allowing us to assess the intrinsic potential of the cells in the absence of HoxB4 transgene expression. FACS analysis of peripheral blood demonstrated multi-lineage donor contributions to both myeloid (GR-1+) and lymphoid (B220, CD4+) compartments. These data demonstrate long-term, multilineage, lymphoid-myeloid hematopoiesis in both primary and secondary animals engrafted with hematopoietic progenitors derived from ES cells by reversible HoxB4 expression.

Applicants have shown that expression of HoxB4 in primitive hematopoietic progenitors from yolk sac or differentiated ES cells, combined with culture on OP9 stroma, promotes the expansion of hematopoietic populations with definitive hematopoictic stem cell potential. The cultured cells express several HSC markers, including c-kit, Sea-1, and CD31, and are rich in hematopoietic colony forming cells, particularly multipotent CFC-GEMMs. In contrast to the embryonic tissues from which they originated, the cultured cells exclusively express the adult isoform of β-globin, as well as CXCR4 and TEL, suggesting that they have undergone a switch from primitive to definitive hematopoietic phenotype. Most importantly, they engraft and repopulate long term lymphoid-myeloid hematopoiesis in irradiated primary and secondary recipients, thereby satisfying the functional definition of the definitive hematopoietic stem cell.

Previous attempts at stable long-term hematopoietic engraftment of adult mice using differentiated ES cells have proven ineffective. The approach described herein represents a less disruptive way to target this cell population, by expressing genes that are normally active in the definitive HSC. HoxB4 is ideal in this regard because its expression confers a competitive advantage on transplanted bone marrow cells without giving rise to leukemia (Sauvageau et al., 1995).

The possible role of HoxB4 in promoting definitive hematopoiesis was suggested by a comparison of Hox gene expression studies, which identify HoxB3, A4, B4, and A5 expression in definitive HSC (Sauvageau et al., 1994), but not in yolk sac (McGrath and Palis, 1997). As defined by extinction of embryonic globin gene expression, and acquisition of adult engraftment and lymphoid-myeloid differentiation potential, Applicants' results suggest that HoxB4 can induce primitive embryonic progenitors to acquire properties characteristic of the adult hematopoietic stem cell. The fact that expression of a single selector gene can promote this switch in pre-circulation yolk sac cells suggests that such cells are poised to become definitive HSC, but whether HoxB4 regulates this fate decision in the embryo is unknown. There is considerable redundancy of function among Hox gene paralogues, and other Hox genes like HoxA4 may be equally capable of promoting this switch. Besides Hox genes, other transcription factors such as CBFA2 may also play a role in specifying definitive hematopoiesis (North et al., 1999). Alternatively, HoxB4 may be acting in a nonphysiological way, by promoting proliferation or enabling the engraftment of a cell that is not normally fated to give rise to definitive hematopoiesis. However, Applicants' attempts to drive long-term engraftment using other growth promoting genes like activated forms of STAT5 and the cytokine receptor c-mpl were unsuccessful, suggesting that this functional potential is specific to HoxB4.

Although in vitro-generated, ES-derived HSCs engraft productively in mice, they reconstitute with a mixture of endogenous and donor-derived hematopoiesis. Thus, additional work remains to understand the competitive profile of ES-derived HSCs compared to their counterparts in fetal liver and adult bone marrow. We have obtained superior lymphoid engraftment from ES cells using inducible HoxB4 expression from the tetracycline response element. In yolk sac cells, retroviral expression of HoxB4 seems to favor myeloid differentiation. A similar effect has been observed in cord blood cells overexpressing HoxB4 (Brun et al., 2001), and with other Hox genes (Buske et al., 2001; Sauvageau et al., 1997). Although transient conditional expression of HoxB4 is superior to constitutive retroviral expression for generating long-term hematopoietic repopulation, the latter is sufficient to enable complete donor hematopoietic chimerism and partial reconstitution of immune function in the immunodeficient mouse model of therapeutic cloning described in the accompanying paper (Rideout et al, 2002).

The classical view of mammalian hematopoietic development held that hematopoietic stem cells originate in the yolk sac, migrate to the fetal liver, and ultimately settle in the bone marrow. More recent work has shown that lymphoid potential and long-term adult-repopulating cells arise at a distinct intraembryonic locale (Cumano et al., 1996; Cumano et al., 2001; Medvinsky and Dzierzak, 1996; Muller et al., 1994; Sanchez et al., 1996), leading to a revised view that primitive and definitive hematopoietic progenitors have distinct origins. Our work and others (Matsuoka et al., 2001; Toles et al., 1989; Weissman et al., 1978; Yoder et al., 1997) shows that primitive embryonic progenitors can contribute to definitive hematopoiesis, suggesting that there may yet be some validity to the classical view. ES cell differentiation recapitulates aspects of both primitive and definitive hematopoiesis in vitro. With the demonstration of hematopoiesis from human ES cells (Kaufman et al., 2001), and a growing interest in therapeutic applications of differentiated cells for regenerative medicine, understanding the key features that distinguish primitive and definitive hematopoiesis may have future clinical significance.

TABLE 1

| | | Live pups (# |
|---|---|---|
| ES Line | # 4n blasts injected | neonatal death) |
| Rag2 −/− | 14 | 4 (0) |
| Rag2 +^{r}/− | 226 | 38 (9) |

Table 1. Mice derived from tetraploid embryo complementation with the Rag2−/− and Rag2+^{R}/− nt ES cell lines

TABLE 2
Surface Antigen Expression of HoxB4-induced ES-derivatives

| Lineage | Antigen | % positive cells |
|---|---|---|
| Myeloid | Gr-1 | 5.6 |
| | Mac-1 | 21.0 |
| Erythroid | Ter119 | 0.7 |
| Lymphoid | B220 | 0.6 |
| | CD4 | 0.0 |
| | CD8 | 0.0 |
| Progenitor/Megakaryocytic | CD41 | 47.8 |
| Pan-hematopoietic | CD45 | 17.0 |
| HSC | c-kit | 80.7 |
| | Sca-1 | 5.4 |
| HSC/Endothelial | CD31 | 78.0 |
| | AA4 | 0.7 |
| | CD34 | 0.5 |
| | Flk-1 | 0.0 |

Table 2. FACS analysis of HoxB4-induced EB-derived cells grown on OP9

### REFERENCES

The following references are cited throughout this application and particularly in Examples 1-5.

Bix, M., Liao, N. S., Zijlstra, M., Loring, J., Jacnisch, R., and Raulet, D. (1991). Rejection of class I MHC-deficient haemopoietic cells by irradiated MHC-matched mice. Nature 349, 329-331.

Brun, A., Fan, X., Humphries, K., and Karlsson, S. (2001). The effects of HOXB4 in human primitive hematopoietic progenitors are concentration dependent: high levels of HOXB4 lead to increased myeloid differentiation and a reduction in proliferative capacity. Blood 98, 66a.

Burdon, T., Stracey, C., Chambers, I., Nichols, J., and Smith, A. (1999). Suppression of SHP-2 and ERK signalling promotes self-renewal of mouse embryonic stem cells. Dev Biol 210, 30-43.

Church, G. M., and Gilbert, W. (1984). Genomic sequencing. Proc Natl Acad Sci U S A 81, 1991-1995.

Cibelli, J. B., Stice, S. L., Golueke, P. J., Kane, J. J., Jerry, J., Blackwell, C., Ponce de Leon, F. A., and Robl, J. M. (1998). Transgenic bovine chimeric offspring produced from somatic cell-derived stem-like cells. Nat Biotechnol 16, 642-646.

Colman, A., and Kind, A. (2000). Therapeutic cloning: concepts and practicalities. Trends Biotechnol 18, 192-196. 8i05_{—}00001434 8i05_{—}00001434.

Cumano, A., Ferraz, J. C., Klaine, M., Di Santo, J. P., and Godin, I. (2001). Intraembryonic, but not yolk sac hematopoietic precursors, isolated before circulation, provide long-term multilineage reconstitution. Immunity 15, 477-485.

Doetschman, T., Gregg, R. G., Maeda, N., Hooper, M. L., Melton, D. W., Thompson, S., and Smithies, 0. (1987). Targetted correction of a mutant HPRT gene in mouse embryonic stem cells. Nature 330, 576-578.

Doevendans, P. A., Kubalak, S. W., An, R. H., Becker, D. K., Chien, K. R., and Kass, R. S. (2000). Differentiation of cardiomyocytes in floating embryoid bodies is comparable to fetal cardiomyocytes. J Mol Cell Cardiol 32, 839-851.

Eggan, K., Akutsu, H., Loring, J., Jackson-Grusby, L., Klemm, M., Rideout, W. M., 3rd, Yanagimachi, R., and Jaenisch, R. (2001). Hybrid vigor, fetal overgrowth, and viability of mice derived by nuclear cloning and tetraploid embryo complementation. Proc Natl Acad Sci U S A 98, 6209-6214.

Hochedlinger, K., and Jaenisch, R. (2002). Monoclonal mice generated by nuclear transfer from mature B and T donor cells. Nature 415, 1035-1038

Hole, N., Graham, G. J., Menzel, U., and Ansell, J. D. (1996). A limited temporal window for the derivation of multilineage repopulating hematopoietic progenitors during embryonal stem cell differentiation in vitro. Blood 88, 1266-1276.

Huang, H., and Auerbach, R. (1993). Identification and characterization of hematopoietic stem cells from the yolk sac of the early mouse embryo. Proc Natl Acad Sci U S A 90, 10110-10114.

Kaufman, D. S., Hanson, E. T., Lewis, R. L., Auerbach, R., and Thomson, J. A. (2001). Hematopoietic colony-forming cells derived from human embryonic stem cells. Proc Natl Acad Sci U S A 98, 10716-10721.

Kawase, E., Yamazaki, Y., Yagi, T., Yanagimachi, R., and Pedersen, R. A. (2000). Mouse embryonic stem (ES) cell lines established from neuronal cell-derived cloned blastocysts. Genesis 28, 156-163.

Kehat, I., Kenyagin-Karsenti, D., Snir, M., Segev, H., Amit, M., Gepstein, A., Livne, E., Binah, O., Itskovitz-Eldor, J., and Gepstein, L. (2001). Human embryonic stem cells can differentiate into myocytes with structural and functional properties of cardiomyocytes. J Clin Invest 108, 407-414.

Keller, G., Kennedy, M., Papayannopoulou, T., and Wiles, M. V. (1993). Hematopoietic commitment during embryonic stem cell differentiation in culture. Mol Cell Biol 13, 473-486.

Kung, S. K., and Miller, R. G. (1995). The NK1.1 antigen in NK-mediated F1 antiparent killing in vitro. J Immunol 154, 1624-1633.

Kyba, M., Perlingeiro, R. C. R., and Daley, G. Q. (2002). Definitive lymphoid-myeloid hematopoiesis from pre-circulation yolk sac and embryonic stem cells. Cell in press.

Laird, P. W., Zijderveld, A., Linders, K., Rudnicki, M. A., Jaenisch, R., and Berns, A. (1991). Simplified mammalian DNA isolation procedure. Nucleic Acids Res 19, 4293.

Lee, L. A., Sergio, J. J., and Sykes, M. (1996). Natural killer cells weakly resist engraftment of allogeneic, long-term, multilineage-repopulating hematopoietic stem cells. Transplantation 61, 125-132.

Lee, S. H., Lumelsky, N., Studer, L., Auerbach, J. M., and McKay, R. D. (2000). Efficient generation of midbrain and hindbrain neurons from mouse embryonic stem cells. Nat Biotechnol 18, 675-679.

Lumelsky, N., Blondel, O., Laeng, P., Velasco, I., Ravin, R., and McKay, R. (2001). Differentiation of embryonic stem cells to insulin-secreting structures similar to pancreatic islets. Science 292, 1389-1394.

Mazurier, F., Fontanellas, A., Salesse, S., Taine, L., Landriau, S., Moreau-Gaudry, F., Reiffers, J., Peault, B., Di Santo, J. P., and de Verneuil, H. (1999). A novel immunodeficient mouse model—RAG2 x common cytokine receptor gamma chain double mutants—requiring exogenous cytokine administration for human hematopoietic stem cell engraftment. J Interferon Cytokine Res 19, 533-541.

Muller, A. M., and Dzierzak, E. A. (1993). ES cells have only a limited lymphopoietic potential after adoptive transfer into mouse recipients. Development 118, 1343-1351.

Munsie, M. J., Michalska, A. E., O'Brien, C. M., Trounson, A. O., Pera, M. F., and Mountford, P. S. (2000). Isolation of pluripotent embryonic stem cells from reprogrammed adult mouse somatic cell nuclei. Curr Biol 10, 989-992.

Nagy, A., Rossant, J., Nagy, R., Abramow-Newerly, W., and Roder, J. C. (1993). Derivation of completely cell culture-derived mice from early-passage embryonic stem cells. Proc Natl Acad Sci U S A 90, 8424-8428.

Notarangelo, L. D., Villa, A., and Schwarz, K. (1999). RAG and RAG defects. Curr Opin Immunol 11, 435-442.

Perlingeiro, R. C., Kyba, M., and Daley, G. Q. (2001). Clonal analysis of differentiating embryonic stem cells reveals a hematopoietic progenitor with primitive erythroid and adult lymphoid-myeloid potential. Development 128, 4597-4604.

Potocnik, A. J., Nerz, G., Kohler, H., and Eichmann, K. (1997). Reconstitution of B cell subsets in Rag deficient mice by transplantation of in vitro differentiated embryonic stem cells. Immunol Lett 57, 131-137.

Reubinoff, B. E., Itsykson, P., Turetsky, T., Pera, M. F., Reinhartz, E., Itzik, A., and Ben-Hur, T. (2001). Neural progenitors from human embryonic stem cells. Nat Biotechnol 19, 1134-1140.

Rideout, W. M., 3rd, Eggan, K., and Jaenisch, R. (2001). Nuclear cloning and epigenetic reprogramming of the genome. Science 293, 1093-1098.

Rozen, S., and Skaletsky, H. J. (1998). Primer3. Code available at http://www-genome.wi.mit.edu/genome software/other/primer3.html.

Sauvageau, G., Thorsteinsdottir, U., Eaves, C. J., Lawrence, H. J., Largman, C., Lansdorp, P. M., and Humphries, R. K. (1995). Overexpression of HOXB4 in hematopoietic cells causes the selective expansion of more primitive populations in vitro and in vivo. Genes Dev 9, 1753-1765.

Sauvageau, G., Thorsteinsdottir, U., Hough, M. R., Hugo, P., Lawrence, H. J., Largman, C., and Humphries, R. K. (1997). Overexpression of HOXB3 in hematopoietic cells causes defective lymphoid development and progressive myeloproliferation. Immunity 6, 13-22.

Schlissel, M. S., Corcoran, L. M., and Baltimore, D. (1991). Virus-transformed pre-B cells show ordered activation but not inactivation of immunoglobulin gene rearrangement and transcription. J Exp Med 172, 711-720.

Shinkai, Y., Rathbun, G., Lam, K. P., Oltz, E. M., Stewart, V., Mendelsohn, M., Charron, J., Datta, M., Young, F., Stall, A. M., and Alt, F. W. (1992). RAG-2-deficient mice lack mature lymphocytes owing to inability to initiate V(D)J rearrangement. Cell 68, 855-867.

Taniguchi, M., Sanbo, M., Watanabe, S., Naruse, I., Mishina, M., and Yagi, T. (1998). Efficient production of Cre-mediated site-directed recombinants through the utilization of the puromycin resistance gene, pac: a transient gene-integration marker for ES cells. Nucleic Acids Res 26, 679-680.

Vogelstein, B., Alberts, B., and Shine, K. (2002). GENETICS: Please Don't Call It Cloning! Science 295, 1237.

Wakayama, T., Perry, A. C., Zuccotti, M., Johnson, K. R., and Yanagimachi, R. (1998). Full-term development of mice from enucleated oocytes injected with cumulus cell nuclei. Nature 394, 369-374.

Wakayama, T., Rodriguez, I., Perry, A. C., Yanagimachi, R., and Mombaerts, P. (1999). Mice cloned from embryonic stem cells. Proc Natl Acad Sci U S A 96, 14984-14989.

Wakayama, T., Tabar, V., Rodriguez, I., Perry, A. C., Studer, L., and Mombaerts, P. (2001). Differentiation of embryonic stem cell lines generated from adult somatic cells by nuclear transfer. Science 292, 740-743.

Wang, Z. Q., Kiefer, F., Urbanek, P., and Wagner, E. F. (1997). Generation of completely embryonic stem cell-derived mutant mice using tetraploid blastocyst injection. Mech Dev 62, 137-145.

Whitehurst, C. E., Chattopadhyay, S., and Chen, J. (1999). Control of V(D)J recombinational accessibility of the D beta 1 gene segment at the TCR beta locus by a germline promoter. Immunity 10, 313-322.

Wiles, M. V., and Keller, G. (1991). Multiple hematopoietic lineages develop from embryonic stem (ES) cells in culture. Development 111, 259-267.

Wilmut, I., Schnieke, A. E., McWhir, J., Kind, A. J., and Campbell, K. H. (1997). Viable offspring derived from fetal and adult mammalian cells. Nature 385, 810-813.

Zhang, S.C., Wernig, M., Duncan, I. D., Brustle, O., and Thomson, J. A. (2001). In vitro differentiation of transplantable neural precursors from human embryonic stem cells. Nat Biotechnol 19, 1129-1133.

The following references are cited throughout the application and particularly in Examples 6-9.

Bronson, S. K., Plaehn, E. G., Kluckman, K. D., Hagaman, J. R., Maeda, N., and Smithies, O. (1996). Single-copy transgenic mice with chosen-site integration. Proc Natl Acad Sci U S A 93, 9067-72.

Brun, A., Fan, X., Humphries, K., and Karlsson, S. (2001). The effects of HoxB4 in human primitive hematopoetic progenitors are concentration dependent: high levels of HoxB4 lead to increased myeloid differentiation and reduction in proliferative capacity. Blood 98, 66a.

Buske, C., Feuring-Buske, M., Antonchuk, J., Rosten, P., Hogge, D. E., Eaves, C. J., and Humphries, R. K. (2001). Overexpression of HOXA10 perturbs human lymphomyelopoiesis in vitro and in vivo. Blood 97, 2286-92.

Cumano, A., Dieterlen-Lievre, F., and Godin, I. (1996). Lymphoid potential, probed before circulation in mouse, is restricted to caudal intraembryonic splanchnopleura. Cell 86.

Cumano, A., Ferraz, J. C., Klaine, M., Di Santo, J. P., and Godin, I. (2001). Intraembryonic, but not yolk sac hematopoietic precursors, isolated before circulation, provide long-term multilineage reconstitution. Immunity 15, 477-85.

Gossen, M., Freundlieb, S., Bender, G., Muller, G., Hillen, W., and Bujard, H. (1995). Transcriptional activation by tetracyclines in mammalian cells. Science 268, 1766-9.

Gould, A., Morrison, A., Sproat, G., White, R. A., and Krumlauf, R. (1997). Positive cross-regulation and enhancer sharing: two mechanisms for specifying overlapping Hox expression patterns. Genes Dev 11, 900-13.

Helgason, C. D., Sauvageau, G., Lawrence, H. J., Largman, C., and Humphries, R. K. (1996). Overexpression of HOXB4 enhances the hematopoietic potential of embryonic stem cells differentiated in vitro. Blood 87, 2740-2749.

Hole, N., Graham, G. J., Menzel, U., and Ansell, J. D. (1996). A limited temporal window for the derivation of multinineage repopulating hematopoietic progenitors during embryonal stem cell differentiation in vivo. Blood 88, 1266-1276.

Kaufman, D. S., Hanson, E. T., Lewis, R. L., Auerbach, R., and Thomson, J. A. (2001). Hematopoietic colony-forming cells derived from human embryonic stem cells. Proc Natl Acad Sci U S A 98, 10716-21.

Keller, G., Kennedy, M., Papayannopoulou, T., and Wiles, M. V. (1993). Hematopoietic Commitment during Embryonic Stem Cell Differentiation in Culture. Mol. Cell. Biol. 13, 473-486.

Matsuoka, S., Tsuji, K., Hisakawa, H., Xu, M.-j., Ebihara, Y., Ishii, T., Sugiyama, D., Manabe, A., Tanaka, R., Ikeda, Y., Asano, S., and Nakahata, T. (2001). Generation of definitive hematopoietic stem cells from murine early yolk sac and paraaortic splanchnopleures by aorta-gonad-mesonephros region-derived stromal cells. Blood 98, 6-12.

McGrath, K. E., and Palis, J. (1997). Expression of Homeobox Genes, Including an Insulin Promoting Factor, in the Murine Yolk Sac at the Time of Hematopoictic Initiation. Mol Reprod Dev 48, 145-153.

Medvinsky, A., and Dzierzak, E. (1996). Definitive hematopoiesis is autonomously initiated by the AGM region. Cell 86, 897-906.

Müller, A. M., and Dzierzak, E. A. (1993). ES cells have only a limited lymphopoietic potential after adoptive transfer into mouse recipients. Development 118, 1343-1351.

Muller, A. M., Medvinsky, A., Strouboulis, J., Grosveld, F., and Dzierzak, E. (1994). Development of hematopoietic stem cell activity in the mouse embryo. Immunity 1, 291-301.

Nakano, T., Kodama, H., and Honjo, T. (1994). Generation of lymphohematopoietic cells from embryonic stem cells in culture. Science 265, 1098-1101.

Naviaux, R. K., Costanzi, E., Haas, M., and Verma, I. M. (1996). The pCL Vector System: Rapid Production of Helper-Free, High-Titer, Recombinan Retroviruses. J. Virol. 70, 5701-5705.

North, T., Gu, T. L., Stacy, T., Wang, Q., Howard, L., Binder, M., Marin-Padilla, M., and Speck, N. A. (1999). Cbfa2 is required for the formation of intra-aortic hematopoietic clusters. Development 126, 2563-75.

Peled, A., Petit, I., Kollet, O., Magid, M., Ponomaryov, T., Byk, T., Nagler, A., Ben-Hur, H., Many, A., Shultz, L., Lider, O., Alon, R., Zipori, D., and Lapidot, T. (1999). Dependence of human stem cell engraftment and repopulation of NOD/SCID mice on CXCR4. Science 283, 845-848.

Perlingeiro, R. C. R., Kyba, M., and Daley, G. Q. (2001). Clonal analysis of differentiating embryonic stem cells reveals a hematopoietic progenitor with primitive erythroid and adult lymphoid-myeloid potential. Development 128, 4597-4604.

Potocnik, a. J., Kohler, H., and Eichmann, K. (1997). Hemato-lymphoid in vivo reconstitution potential of subpopulations derived from in vitro differentiated embryonic stem cells. Proc. Natl. Acad. Sci. USA 94, 10295-10300.

Sanchez, M. J., Holmes, A., Miles, C., and Dzierzak, E. (1996). Characterization of the first definitive hematopoietic stem cells in the AGM and liver of the mouse embryo. Immunity 5, 513-25.

Sauvageau, G., Landsdorp, P. M., Eaves, C. J., Hogge, D. E., Dragowska, W. H., Reid, D. S., Largman, C., Lawrence, H. J., and Humphries, R. K. (1994). Differential expression of homeobox genes in functionally distinct CD34⁺ subpopulations of human bone marrow cells. Proc. Natl. Acac. Sci. USA 91, 12223-12227.

Sauvageau, G., Thorsteinsdottir, U., Eaves, C. J., Lawrence, H. J., Largman, C., Lansdorp, P. M., and Humphries, R. K. (1995). Overexpression of HOXB4 in hematopoietic cells causes the selective expansion of more primitive populations in vitro and in vivo. Genes Dev. 9, 1753-1765.

Sauvageau, G., Thorsteinsdottir, U., Hough, M. R., Hugo, P., Lawrence, H. J., Largman, C., and Humphries, R. K. (1997). Overexpression of HOXB3 in hematopoietic cells causes defective lymphoid development and progressive myeloproliferation. Immunity 6, 13-22.

Toles, J. F., Chui, D. H., Belbeck, L. W., Starr, E., and Barker, J. E. (1989). Hemopoietic stem cells in murine embryonic yolk sac and peripheral blood. Proc Natl Acad Sci U S A 86, p7456-9.

Van Parijs, L., Refaeli, Y., Lord, J. D., Nelson, B. H., Abbas, A. K., and Baltimore, D. (1999). Uncoupling IL-2 signals that regulate T cell proliferation, survival, and Fas-mediated activation-induced cell death. Immunity 11, 281-8.

Wang, L. C., Swat, W., Fujiwara, Y., Davidson, L., Visvader, J., Kuo, F., Alt, F. W., Gilliland, D. G., Golub, T. R., and Orkin, S. H. (1998). The TEL/ETV6 gene is required specifically for hematopoiesis in the bone marrow. Genes Dev 12, 2392-2402.

Weissman, I., Papaioannou, V., and Gardner, R. (1978). Fetal hematopoietic origins of the adult hematolymphoid system. In Differentiation of normal and neoplastic hematopoietic cells, C. B, M. PA and T. JE, eds. (Cold Spring Harbor, N.Y.: Cold Spring harbor Laboratory), pp. 33-47.

Wutz, A., Rasmussen, T. P., and Jaenisch, R. (2002). Chromosomal silencing and localization are mediated by different domains of Xist RNA. Nat Genet 30, 167-174.

Yoder, M. C. (2001). Introduction: spatial origin of murine hematopoictic stem cells. Blood 98, 3-5.

Yoder, M. C., Hiatt, K., Dutt, P., Mukherjee, P., Bodine, D. M., and Orlic, D. (1997). Characterization of definitive lymphohematopoictic stem cells in the day 9 murine yolk sac. Immunity 7, 335-344.

Zambrowicz, B. P., Imamoto, A., Fiering, S., Herzenberg, L. A., Kerr, W. G., and Soriano, P. (1997). Disruption of overlapping transcripts in the ROSA beta geo 26 gene trap strain leads to widespread expression of beta-galactosidase in mouse embryos and hematopoietic cells. Proc Natl Acad Sci U S A 94, 3789-94.

While this invention has been particularly shown and described with reference to preferred embodiments thereof, it will be understood by those skilled in the art that various changes in form and details may be made therein without departing from the spirit and scope of the invention as defined by the appended claims. Those skilled in the art will recognize or be able to ascertain, using no more than routine experimentation, many equivalents to the specific embodiments of the invention described herein. Such equivalents are intended to be encompassed in the scope of the claims. The entire teachings of all references cited herein are incorporated by reference into this application.

We claim:

1. A method of correcting a genetic defect in an individual in need thereof, comprising administering to the individual a therapeutically effective amount of repaired ntES cells and/or repaired differentiated progenitor or precursor cells derived from repaired ntES cells, wherein in the repaired ntES cells, the genetic defective has been corrected.

2. The method of claim 1, wherein the genetic defect was corrected in ntES cells by a recombinant nucleic acid method.

3. The method of claim 2, wherein the recombinant nucleic acid method is a recombinant DNA method.

4. The method of claim 3, wherein the recombinant nucleic acid method is homologous recombination.

5. The method of claim 4, wherein homologous recombination occurs between (a) DNA in ntES cells which comprises the genetic defect to be corrected and (b) DNA that (i) is introduced into the ntES cells; (ii) comprises DNA that, when introduced into DNA in the ntES cells corrects the genetic defect; and (iii) undergoes homologous recombination with DNA in the ntES cells in such a manner that the genetic defect is corrected, thereby correcting the genetic defect in the ntES cells and resulting in production of repaired ntES cells.

6. The method of claims 1, wherein the genetic defect is selected from the group consisting of: a genetic defect that causes an immune system disorder; a genetic defect that causes a neurological disorder; a genetic defect that causes a cardiac disorder; a genetic defect that causes a circulatory disorder and a genetic defect that causes a respiratory disorder.

7. A method of treating a genetic disorder in an individual in need thereof, comprising administering to the individual a therapeutically effective amount of repaired ntES cells and/or repaired differentiated progenitor or precursor cells derived from repaired ntES cells, wherein in the repaired cells, a defect in a gene or genes that causes or is associated with the genetic disorder has been corrected.

8. The method of claim 7, wherein the genetic defect has been corrected by a recombinant nucleic acid method.

9. The method of claim 8, wherein the recombinant nucleic acid method is a recombinant DNA method.

10. The method of claim 9, wherein the recombinant nucleic acid method is homologous recombination.

11. The method of claim 10, wherein homologous recombination occurs between (a) DNA in ntES cells which comprises the genetic defect to be corrected and (b) DNA that (i) is introduced into the ntES cells; (ii) comprises DNA that, when introduced into DNA in the ntES cells corrects the genetic defect; and (iii) undergoes homologous recombination with DNA in the ntES cells in such a manner that the genetic defect is corrected, thereby correcting the genetic defect in the ntES cells.

12. The method of claim 7, wherein the genetic disorder is selected from the group consisting of: an immune system disorder; a neurological disorder; a cardiac disorder; a circulatory disorder and a respiratory disorder.

13. Repaired ntES cells.

14. Repaired ntES cells of claim 13, wherein the cells are mammalian cells.

15. Repaired ntES cells of claim 14, wherein the mammalian cells are human cells or mouse cells.

16. A method of producing repaired ntES cells, comprising:
(a) introducing nuclei from a somatic cell into enucleated oocytes, wherein the somatic cell comprises DNA comprising a genetic defect;
(b) maintaining the product of (a) under conditions appropriate for blastocyst formation, thereby producing blastocysts comprising DNA from the somatic cell;
(c) obtaining embryonic stem cells from blastocysts produced in (b), wherein the embryonic stem cells are referred to as ntES cells and comprise DNA comprising the genetic defect; and
(d) correcting the genetic defect in the ntES cells, thereby producing repaired ntES cells.

17. The method claim 16, wherein the ntES cells are mouse cells.

18. The method of claim 16, wherein the ntES cells are human cells.

19. The method of claim 16, wherein the genetic defect is corrected in ntES cells by a recombinant nucleic acid method.

20. The method of claim 19, wherein the recombinant nucleic acid method is a recombinant DNA method.

21. The method of claim 20, wherein the recombinant nucleic acid method is homologous recombination.

22. In this method, the genetic defect can be corrected in ntES cells by a recombinant nuclei acid method (e.g., homologous recombination). In this embodiment, homologous recombination occurs between (a) DNA in ntES cells which comprises the genetic defect to be corrected and (b) DNA that (i) is introduced into the ntES cells; (ii) comprises DNA that, when introduced into DNA in the ntES cells corrects the genetic defect; and (iii) undergoes homologous recombination with DNA in the ntES cells in such a manner that the genetic defect is corrected, thereby correcting the genetic defect in the ntES cells and resulting in production of repaired ntES cells.

23. The genetic defect corrected can be, for example, a genetic defect that causes an immune system disorder; a genetic defect that causes a neurological disorder; a genetic defect that causes a cardiac disorder; a genetic defect that causes a circulatory disorder or a genetic defect that causes a respiratory disorder.
